SEQUENCE LISTING (6 sequences)

SEQ ID NO: 1 - DNA, 2059 bp, Homo sapiens
  CDS (17..1381, 1409..2050)
tctagaggagggatttatgggggtccagcttcagcagtcaggacctgacctggtgaaacctggggcctcagtgaagatatcctgcaaggcttctggatacacattcactgactacaacatacactgggtgaagcagagccgtggaaagagccttgagtggattggatatatttatccttacaatggtaatacttactacaaccagaagttcaagaacaaggccacattgactgtagacaattcctccacctcagcctacatggagctccgcagcctgacatctgaggactctgcagtctattactgtgcaacctactttgactactggggccaaggcaccactctcacagtgagctcagcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagaacgtgaccgccagaaacttcccacctagccaggatgcctccggggacctgtacaccacgagcagccagctgaccctgccggccacacagtgcccagacggcaagtccgtgacatgccacgtgaagcactacacgaattccagccaggatgtgactgtgccctgccgagttcccccacctcccccatgctgccacccccgactgtcgctgcaccgaccggccctcgaggacctgctgttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtcatcagtacttcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccacctatgctgtgaccagcatactgcgcgtggcagccgaggactggaagaagggggagaccttctcctgcatggtgggccacgaggccctgccgctggccttcacacagaagaccatcgaccgcttggcgggtaaacccacccatatcaatgtgtctgttgtcatggcggaggcggacggcacctgctacagatgaaatattgcggatccggagggatttatggacattgtgatgacccagtctccagcaatcatgtctgcatctccaggggagaaggtcaccataacctgcagtgccagctcaatggtaagttacatgcactggttccagcagaagccaggcacttctcccaaactctggctttatagcacatccaacctggcttctggagtccctgctcgcttcagtggcagtggatctgggacctcttactctctcacaatcagccgaatggaggctgaagatgctgccacttattactgccatcaaaggactagctacccatacacattcggaggggggaccaagcttgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatctaga

SEQ ID NO: 2 - protein, 669 aa, Homo sapiens
MGVQLQQSGPDLVKPGASVKISCKASGYTFTDYNIHWVKQSRGKSLEWIGYIYPYNGNTYYNQKFKNKATLTVDNSSTSAYMELRSLTSEDSAVYYCATYFDYWGQGTTLTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRLAGKPTHINVSVVMAEADGTCYRMDIVMTQSPAIMSASPGEKVTITCSASSMVSYMHWFQQKPGTSPKLWLYSTSNLASGVPARFSGSGSGTSYSLTISRMEAEDAATYYCHQRTSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 50 bp, Artificial Sequence
  Description of Artificial Sequence Primer
aaaatctagaggagggatttatgcagacatctgtgtccccctcaaaagtc

SEQ ID NO: 4 - DNA, 37 bp, Artificial Sequence
  Description of Artificial Sequence Primer
cataccggggactagtcacattcacggtcacctcgcg

SEQ ID NO: 5 - protein, 5 aa, Artificial Sequence
  Description of Artificial Sequence Synthetic peptide
MQTSV

SEQ ID NO: 6 - protein, 4 aa, Artificial Sequence
  Description of Artificial Sequence Synthetic peptide
KDEL

(19) United States
(12) Patent Application Publication
(10) Pub. No.: US 2006/0282920 A1
(43) Pub. Date: Dec. 14, 2006

(54) Production of antibodies in transgenic plastids

(76) Inventor: Henry Daniell, Winter Park, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/352,733

(22) Filed: Feb. 13, 2006

Related U.S. Application Data
(63) Continuation of application No. 09/807,721, filed on Apr. 18, 2001, now abandoned.
(60) Provisional application No. 60/185,661, filed on Feb. 29, 2000.

(51) Int. Cl. C12N 15/82

(52) U.S. Cl. 800/288; 435/320.1; 800/278

(57) ABSTRACT

This invention provides compositions and methods for the transformation of plastids of plant cells with multiple genes, and proper association or assembly of multimeric proteins that are heterologous to the plastids of plant cells. A plasmid construct encoding all of the individual polypeptide components of the multimeric protein is provided. Stable integration of the heterologous coding sequences into the plastid genome of the target plant is accomplished through homologous recombination. The present invention achieves assembly of immunoglobulin heavy and light chains, with covalent bonding between the chains, into immunologically active immunoglobulins in the chloroplast.

### RELATED APPLICATIONS

This application is a continuation of U.S. application Ser. No. 09/807,721, filed Apr. 18, 2001; which is the National Stage of International Application No. PCT/US01/06274, filed Feb. 28, 2001; which claims the benefit of U.S. Provisional Application No. 60/185,661, filed Feb. 29, 2000, all of which are hereby incorporated by reference herein in their entirety.

### TECHNICAL FIELD

This invention relates to compositions and methods for production of multimeric proteins, including antibodies, in plants containing transformed plastids.

### BACKGROUND

Using transgenic plants to produce industrial or therapeutic biomolecules is one of the fastest developing areas in biotechnology. Recombinant proteins like monoclonal antibodies, vaccines, hormones, growth factors, neuropeptides, cytotoxins, serum proteins and enzymes have been expressed in nuclear transgenic plants (May et al., 1996).

Plants provide several advantages for the production of therapeutic proteins, including lack of contamination with animal pathogens, relative ease of genetic manipulation, eukaryotic protein modification machinery and economical production. Plant genetic material is indefinitely stored in seeds, which require little or no maintenance. In particular, transgenic plants offer a number of advantages for production of recombinant/monoclonal antibodies. Plants have no immune system, therefore only one antibody species is expressed, and the absence of mammalian viruses and other pathogens provides maximum safety for humans and animals. Some types of monoclonal antibodies, such as secretory IgA (SIgA) can be produced in large quantities only in plants (Ma et al., 1995).

The first report of antibodies produced in plants (plantibodies) was published by Hiatt in 1989 (Hiatt et al., 1989) and subsequently by many others (Düring et al., 1990; Ma et al., 1998; Ma et al., 1995; Ma et al., 1994; Verch et al., 1998; Zeitlin et al., 1998). Sexual crosses between plants individually expressing immunoglobulin heavy and light chains are the classical method to obtain transgenic plants expressing full length assembled antibody. This method, however, is time consuming. An alternative method is co-transformation with two different Agrobacterium strains, one carrying heavy and one carrying light chain, along with two different selectable markers, although efficiency of co-transformation is low (De Neve, et al., 1993). Expression and assembly of a full-length monoclonal antibody (mAb) in Nicotiana benthamina plants using a plant virus vector has also been reported (Verch et al., 1998).

Despite the many attractive features of current plant expression systems, however, a major limitation in producing antibodies in plants has been their generally low level of expression. The highest accumulation levels reported for full-size antibodies in plants are less than 1% of total soluble protein (DeNeve et al., 1999; Ma et al., 1994; van Engelen et al., 1994). Levels as high as 5% to 6% have been reported for secretory IgA (SIgA) (Ma et al., 1995) and for single chain antibodies (ScFv) (Artsaenko et al., 1995; Fiedler et al., 1997). However, these numbers probably include non-functional antibody. Our experience with SIgA-producing plants (Ma et al, 1995) has taught us that levels of functional antibody in a recoverable form are much lower than the total amount of antibody that can be detected by western blotting. The highest yield of soluble, functional antibody from transgenic tobacco was 10-80 mg/kg fresh weight of transgenic leaves (Ma et al., 1998). This may reflect, in part, an insolubilization of antibody in the apoplastic space when secreted from the plant cell. In addition, a phenomenon known as post-transcriptional gene silencing may place an upper limit on the expression of nuclear transgenes in plants, including antibody genes (Vaucheret et al., 1998; De Neve et al., 1999; Wycoff, unpublished results). Novel means of generating very high antibody expression in plants are likely to make the commercial use of transgenic plants highly attractive and competitive.

Another impediment to producing antibodies in plants is the environmental concerns of nuclear genetic engineering. Despite the widespread planting of genetically engineered crops in the U.S. (nearly 50% of corn, cotton and soybean planted in the U.S. are now genetically modified), environmental concerns have led to wariness and a lack of acceptance by part of the public of genetically modified (GM) crops around the world (Daniell, 1999a-d). One common environmental concern is the escape of foreign genes through pollen or seed dispersal, thereby creating super weeds or causing genetic pollution among other crops. If significant rates of such gene flow are generally shown from crops to wild relatives (as high as 38% in sunflower and 50% for strawberries) there may be cause for serious concern. In addition, allegations of genetic pollution among crops have resulted in several lawsuits and shrunk the European market for organic produce from Canada from 83 tons in 1994-1995 to 20 tons in 1997-1998 (Hoyle, 1999). Another environmental concern expressed recently is the possibility of toxicity of transgenic pollen from plants modified to express the insecticidal protein of Bacillus thuringensis (B.t.) to non-target insects, including Monarch butterflies (Losey et al., 1999), although more recent studies indicate this is not a significant problem (Niller, 1999). Yet another environmental concern has been the development of insects resistant to the insecticidal protein B.t., due to low levels (sub-lethal) of nuclear expression in transgenic plants (Gould, 1998).

An alternative to nuclear transformation of plants that may address both productivity and environmental concerns is the expression of proteins such as antibodies in plastids. The advantages of plastids over nuclear transformants have been summarized in several recent reviews (Daniell, 1999A-D). Plastids are maternally inherited and are not transferred through pollen (Scott and Wilkinson, 1999). This has been clearly demonstrated using a herbicide resistance gene introduced via plastid genetic engineering (Daniell et al., 1998). Thus gene flow due to the presence of a transgene in pollen, is not a problem with plastid transformation. The plastid is also a protein factory par excellence: most of the protein in a typical leaf cell is found in plastids. Hyper-expression of foreign proteins (up to 47% of total soluble protein) has been accomplished via plastid genetic engineering (DeCosa et al., 2001). Comparisons between nuclear and plastid expression of the same transgene have shown that expression in plastids exceeds, by many-fold that from the nucleus. For example, biologically active recombinant human somatotropin, including the appropriate disulfide bonds, has recently been expressed in plastids at levels of up to 7% of total soluble protein (Staub et al., 2000). This level of somatotropin in plastids was 300-fold higher than levels in the best transgenic plants expressing somatotropin from a nuclear transgene.

Early investigations in plastid genetic engineering involved introduction of isolated plastids expressing foreign genes into protoplasts (Carlson, 1973, Daniell et al., 1986, Daniell and McFadden, 1987). However, after discovery of the Gene Gun, transient foreign gene expression in dicots (Daniell et al., 1990, Ye et al., 1990) and monocots (Daniell et al., 1991) was followed by stable foreign gene expression. Plants resistant to B.t. resistant insects (up to 40,000 fold) were obtained by hyperexpression of the cryIIA gene (Kota et al., 1999). Plants were also genetically engineered via the plastid genome to confer herbicide resistance; introduced foreign genes were maternally inherited, overcoming the problem of out-cross with weeds or other crops (Daniell et al. 1998). Plastid genetic engineering has been used to produce pharmaceutical proteins (Guda et al. 1999). Plastid genetic engineering is now extended to other useful crops (Sidorov et al. 1999; Daniell, 1999E). Nevertheless there has, until now, not been a demonstration of expression and assembly of an antibody in transgenic plastids.

Compartmentalization of foreign proteins in plastids facilitates their purification. Intact plastids are easy to isolate from crude homogenates by low-speed centrifugation and may be burst open by osmotic shock to release foreign proteins that are compartmentalized within (Daniell and McFadden, 1987). Another advantage of plastids is that they can efficiently translate polycistronic messages (Daniell et al., 1994). Antibody heavy and light chains (and other proteins if desired) can be introduced into a single site in the plastid genome, although functional expression of multimeric proteins have not been shown until the present invention.

Plastids do not glycosylate their proteins. Although glycosylation is required for complement binding and effector function for some antibodies in serum, the effectiveness of antibodies at mucosal surfaces does not appear to involve glycosylation. Many single chain Ab fragments (scFv) and Fab's entirely lacking the constant regions of Ab molecule where glycosylation occurs bind to their appropriate antigen with the same affinity as the native Ab (Boss, et al., 1984). Antibodies made in plastids may have advantages for parenteral (injectable) uses, since they will not carry the potentially immunogenic plant N-linked glycans found on nuclear-encoded plantibodies.

In summary, the plastid genome is thus an attractive target for introduction and expression of antibody genes. The reasons include: 1) capacity for extraordinarily high levels of foreign protein expression, 2) ability to fold, process and assemble eukaryotic proteins, 3) simpler purification, 4) containment of foreign genes through maternal inheritance and 5) no glycosylation.

Despite the potential advantages of plastids for antibody production, it was not obvious that antibodies expressed in plastids would assemble in this organelle. Assembled antibody was detected in plastids of transgenic tobacco (Düring et al., 1990), but the plastids themselves were not transformed and neither heavy nor light chain of the antibody could be recovered from the cell. Prior to this patent application there were no published reports of expression of antibodies in plastids, and there were valid reasons to suggest that it would be problematic. In mammalian plasma cells the immunoglobulin light and heavy chains, encoded by nuclear genes, are synthesized as precursor proteins containing an amino-terminal signal peptide that guides the chains into the lumen of the endoplasmic reticulum (ER). The signal peptide is cleaved off in the ER and stress proteins such as BiP/GRP78 and GRP94, which function as chaperoning, bind to unassembled light and heavy chains and direct their folding and assembly (Gething and Sambrook., 1992; Melnick et al., 1992). Disulfide bond formation is catalyzed by protein disulfide isomerase and N-linked glycans are attached in the ER and further processed in the Golgi, before the antibody is secreted from the cell.

This process appears to be broadly similar in nuclear transgenic plants (Hiatt et al., 1989), where homologues to the chaperonins BiP and GRP94 have been reported (Fontes et al., 1991; Walther-Larsen et al., 1993). Even so, there was no certainty that antibody heavy and light chains would assemble normally in plastids, or that they would retain their antigen-binding activity. There might have been unforeseen deleterious effects of high-level expression of antibodies in plastids on plant growth or development that were not apparent from the experiences with other transgenes. The pH and oxidation state of the plastid differs from that of the ER in ways that might inhibit or prevent antibody folding and assembly.

On the other hand, it has been known for some time that disulfide bonds exist both within (Ferri et al., 1978) and between some plastid proteins (Ranty et al., 1991; Schreuder et al., 1993; Drescher et al., 1998). Both nuclear and plastid encoded proteins are activated by disulfide bond oxidation/reduction cycles using the plastid thioredoxin system (Ruelland and Miginiac-Maslow, 1999) or plastid protein disulfide isomerase (Kim and Mayfield, 1997). Chaperonin molecules of the HSP70 and HSP60 families, including the rubisco binding protein, have also been reported in plastids (Roy, 1989; Vierling, 1991). These molecules function in the folding and assembly of eukaryotic (nuclear) and prokaryotic (plastid) proteins. We hypothesized that they would be able to assist in the proper assembly of immunoglobulin chains in plastids.

There are examples of protein complexes in the plastid in which all the subunits are native to the plant, the ribosome being an example. However, the expression and assembly in transformed plastids of heterologous proteins into multi-protein complexes has not been reported until the present invention. There is a single example in the literature of an inter-chain disulfide bond in plant plastids, and that is between neighboring large subunits of the enzyme ribulose-1, 5-biphosphase carboxylase/oxygenase (Ranty et al., 1991). The expression and assembly in transformed plastids of functional proteins consisting of different protein chains, including disulfide bonds between different subunits, as represented by expression and assembly of a mammalian antibody has never been demonstrated until the present invention.

### SUMMARY OF THE INVENTION

The present invention provides compositions and methods for the transformation of plastids of plant cells with multiple genes, and proper association or assembly of multimeric proteins that are heterologous to be plastids of plant cells. A plasmid construct encoding all of the individual polypeptide components of the multimeric protein is used. Typically, the plasmid used in the invention is made as an “expression cassette” which includes regulatory sequences. For example an expression cassette might include, operationally joined, DNA sequences coding for immunoglobulin heavy and light chains separated by a small linker containing an intervening stop codon and ribosome binding site, and control sequences positioned upstream from the 5′ and downstream from the 3′ ends of the coding sequences to provide expression of the coding sequences in the plastid genome. Flanking each side of this expression cassette would be DNA sequences that are homologous to a sequence of the target plastid genome. Stable integration of the heterologous coding sequences into the plastid genome of the target plant is accomplished through homologous recombination. The present invention achieves assembly of immunoglobulin heavy and light chains, with covalent bonding between the chains, into immunologically active immunoglobulins in the plastid.

Alternatively, the expression cassette may include, operationally joined, DNA sequences coding for J chain and Secretory Components separated by a small linker containing an intervening stop codon and ribosome binding site, and control sequences positioned upstream from the 5′ and downstream from the 3′ ends of the coding sequences to provide expression of these coding sequences in the plastid genome. Homologous flanking sequences that may be the same as or different than the ones provided for the expression cassette containing the immunoglobulin heavy and light chains are similarly provided for this cassette. In addition to assembly of the immunologically active immunoglobulins in the plastid, Secretory Component and J chain are also assembled with the immunoglobulin, when the heavy chain is an α (alpha) chain thereby producing secretory immunoglobulin A (SIgA).

The antibodies produced by the present invention are antibodies which are useful for mammals, including animals and human, where it is generally accepted in the art to use antibodies in therapy.

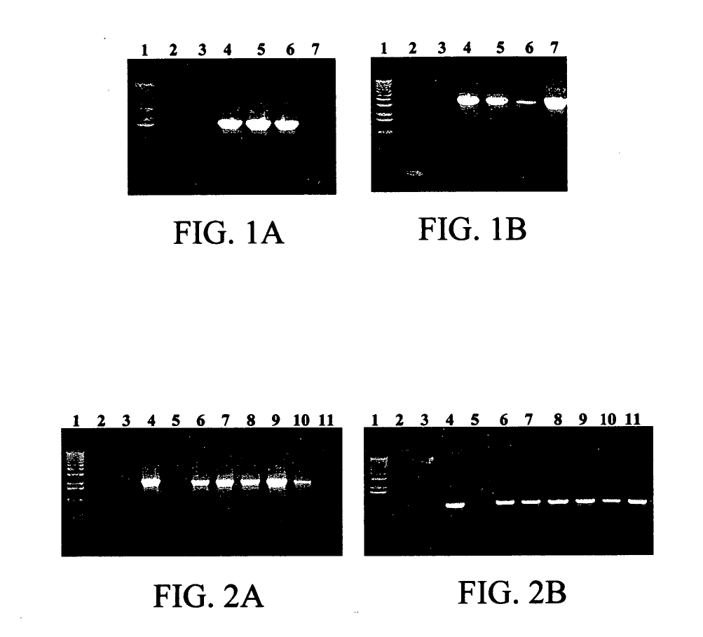

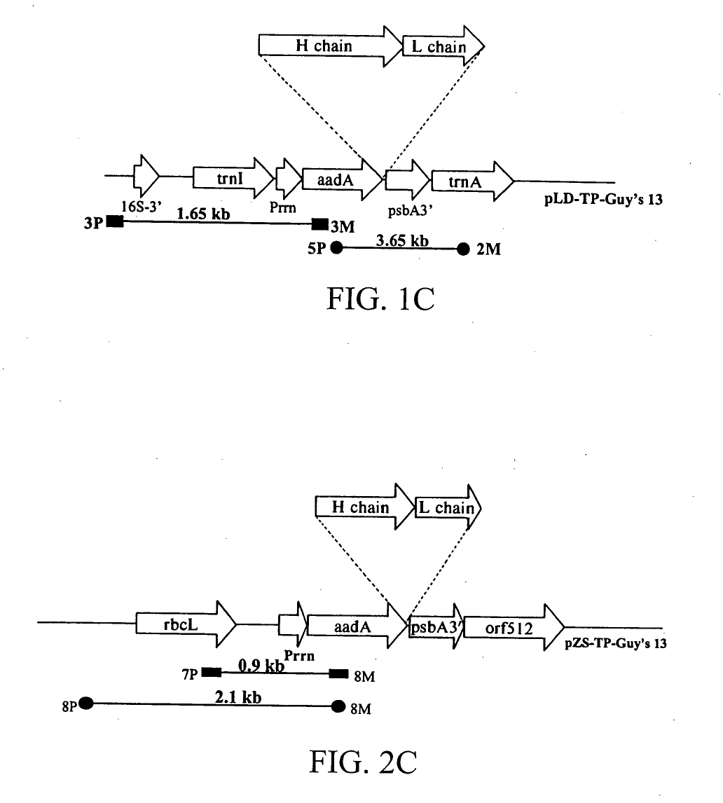

FIG. 1. Construction of the pLD-TP-Guy's 13 vector and PCR analysis of spectinomycin-resistant tobacco clones transformed with pLD-TP-Guy's 13. FIG. 1A. PCR analysis to show integration of the aadA gene, using the 3P and 3M primer pair. FIG. 1B. PCR analysis to show integration of the H and L immunoglobulin genes, using the 5P and 2M primer pair. FIG. 1C. The plastid vector pLD-TP-Guy's 13 and primer annealing sites. Lane 1, 1 kb ladder; Lane 2, negative control without template; Lane 3, negative control untransformed plant; Lanes 4-6, transformed plants; Lane 7, the plasmid pLD-TP-Guy's 13.

FIG. 2[A]. Construction of the pZS-TP-Guy's 13 vector and PCR analysis of spectinomycin resistant clones transformed with pZS-TP-Guy's 13. FIG. 2A. PCR analysis of spectinomycin-resistant tobacco clones using 8P and 8M primer pair. FIG. 2B. PCR analysis of spectinomycin-resistant tobacco clones using 7P and 8M primer pair. FIG. 2C. The plastic pZS-TR Guy's 13 and primer annealing sites. Lane 1, 1 kb ladder; Lane 2, negative control without template; Lane 3, negative control untransformed plant; Lane 4, positive control previously characterized pZS-transformed plant; Lane 5, mutant clone; Lanes 6-10, transformed clones; Lane 11, the plasmid pZS-TP-Guy's 13.

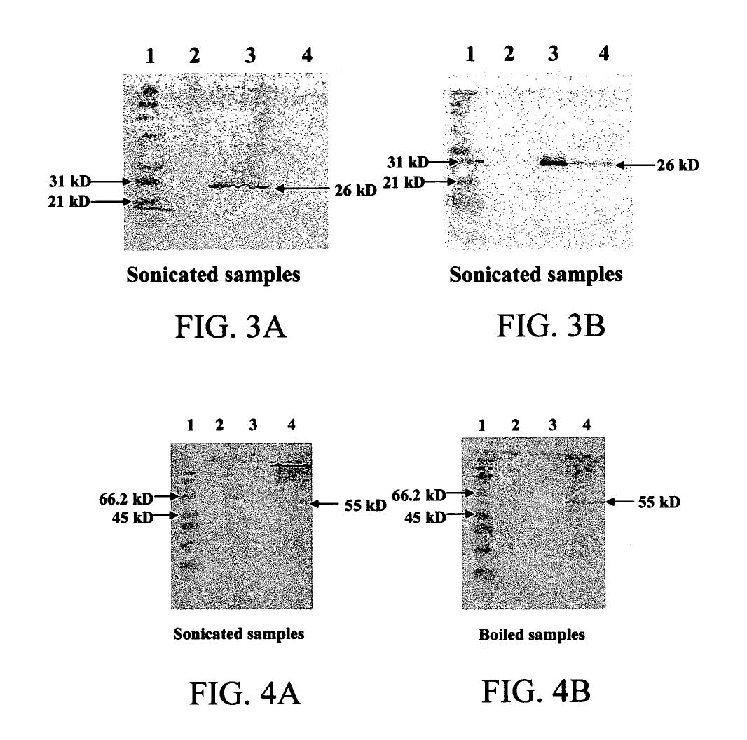

FIGS. 3A and 3B. Western blot analysis of antibody light chain expression in [E. coli] E. coli by the tobacco and universal vectors: FIG. 3A, Lane 1, molecular weight markers; Lane 2, negative control (insert in the wrong orientation); Lane 3A, XL1-Blue cells transformed with the pZS-TP-Guy's 13 vector; Lane 4A, negative control (untransformed XL1-Blue cells); [Lane] FIG. 3B, positive control Human IgA; Lane 4B, XL1-Blue cells transformed with the pLD-TP-Guy's 13 vector. Blots were probed with AP-conjugated goat anti-human kappa antibody.

FIGS. 4A and 4B. Western blot analysis of antibody heavy chain expression in E. coli by the tobacco vector. Lane 1, molecular weight markers; Lane 2, negative control (insert in the wrong orientation); Lane 3, negative control (untransformed XL1-Blue cells); Lane 4, XL1-Blue cells transformed with the pZS-TP-Guy's 13 vector. Samples in [blot A] FIG. 4A were sonicated, and those in [blot B] FIG. 4B were boiled. Blots were probed with Ap-conjugated goat anti-human IgA antibody.

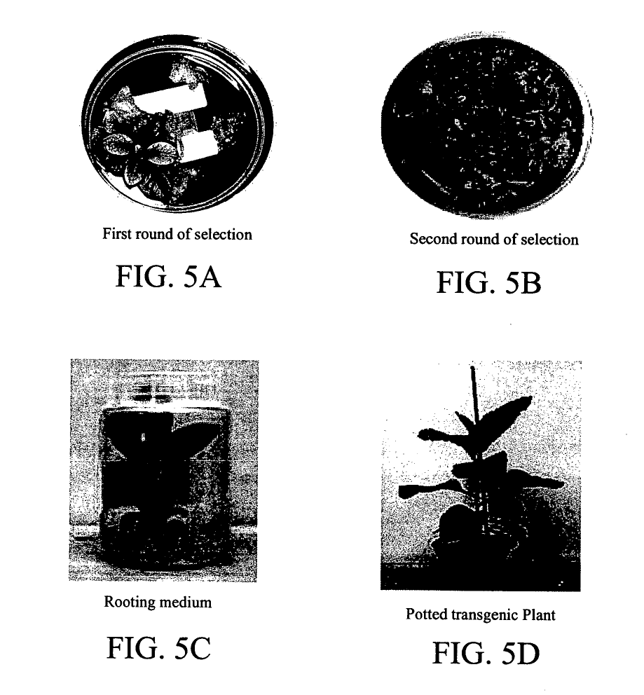

FIGS. 5A-5D. Steps in plastid transformation and regeneration of plastid transgenic plants.

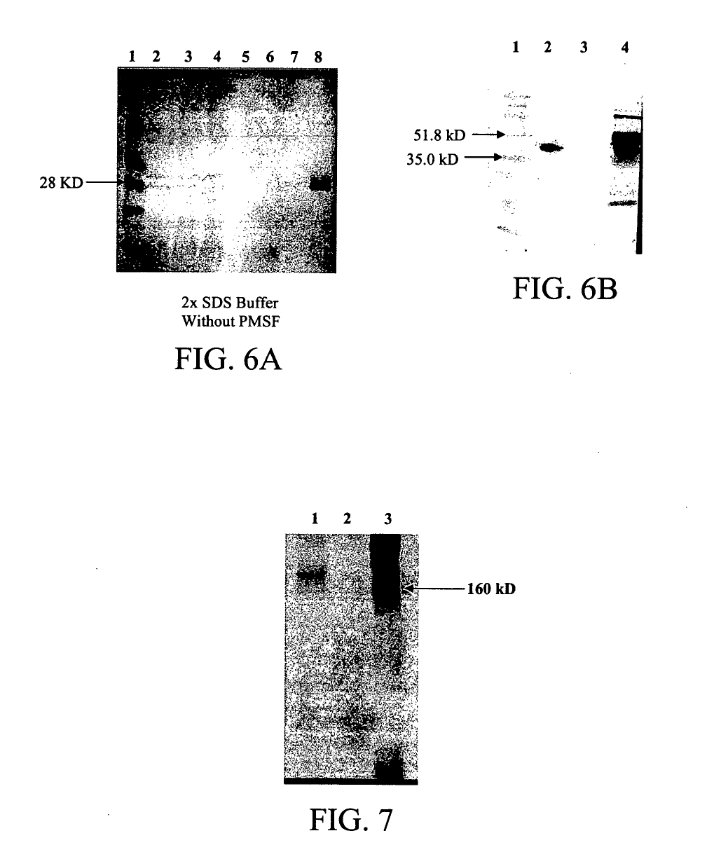

FIGS. 6A and 6B. Western blot analysis of antibody expression in Tobacco plastids. Lane 1, molecular weight markers; Lanes 2-4, extracts from different transgenic plants; Lanes 5 and 7, blank[,]; Lane 6, negative control extract from an untransformed plant; Lane 8, positive control human IgA. The gels were run under non-reducing conditions. [Blot A]FIG. 6A was developed with AP-conjugated goat anti-human kappa antibodies. [Blot B]FIG. 6B was developed using AP-conjugated goat anti-human IgA antibodies.

FIG. 7. Western blot analysis of transgenic lines showing the assembled antibody. Lanes 1 and 2, extracts from transgenic plants; Lane 3, negative control extract from an untransformed plant; Lane 4 positive control human IgA. The gel was run under non-reducing conditions, and the blot was developed with AP-conjugated goat anti-human kappa antibody.

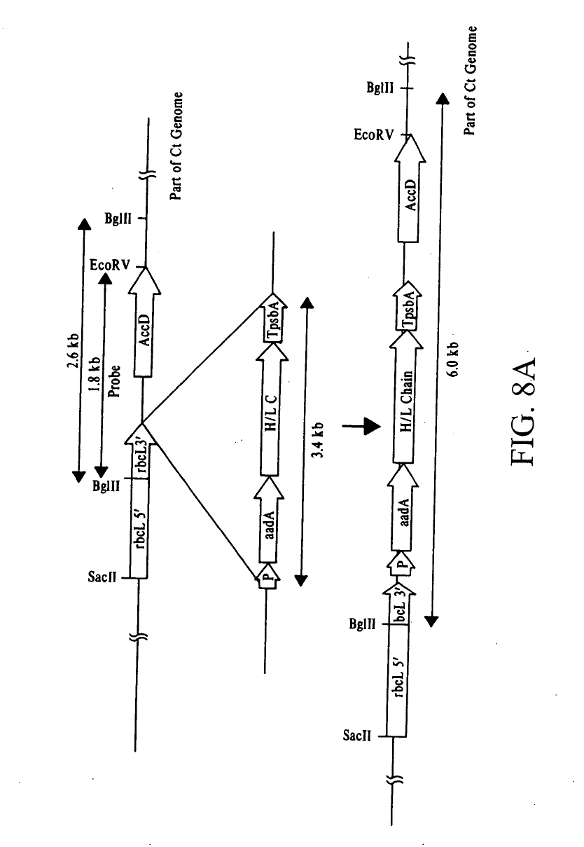

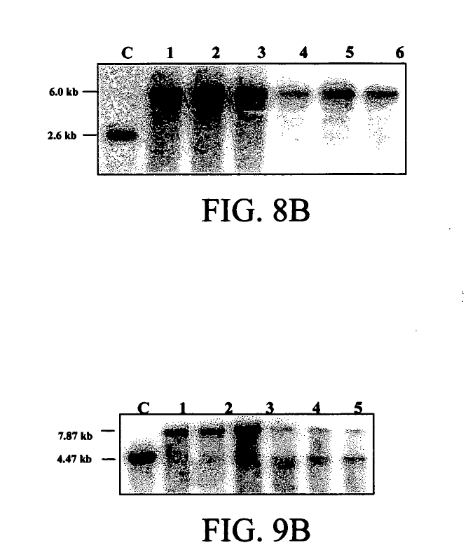

FIGS. 8A and 8B. FIG. 8A: Southern blot analysis of the clones transformed with the pZS-TP-Guy's 13 vector. FIG. 8B, Lane C, control untransformed Petit Havana; Lanes 1-6, transgenic lines.

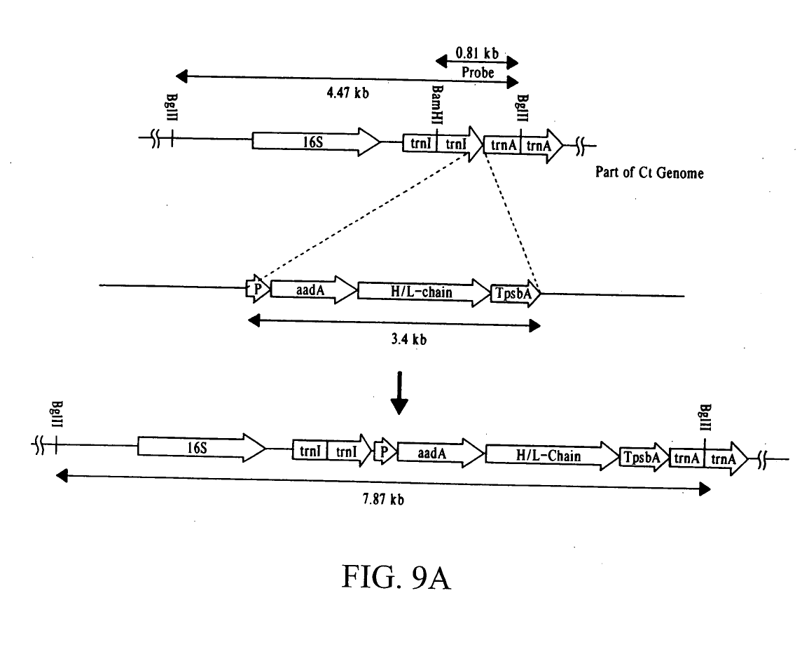

FIGS. 9A and 9B. FIG. 9A: Southern blot analysis of the clones transformed with the pLD-Guy's 13 vector. FIG. 9B, Lane C, control untransformed Petit Havana; Lanes 1-6, transgenic lines.

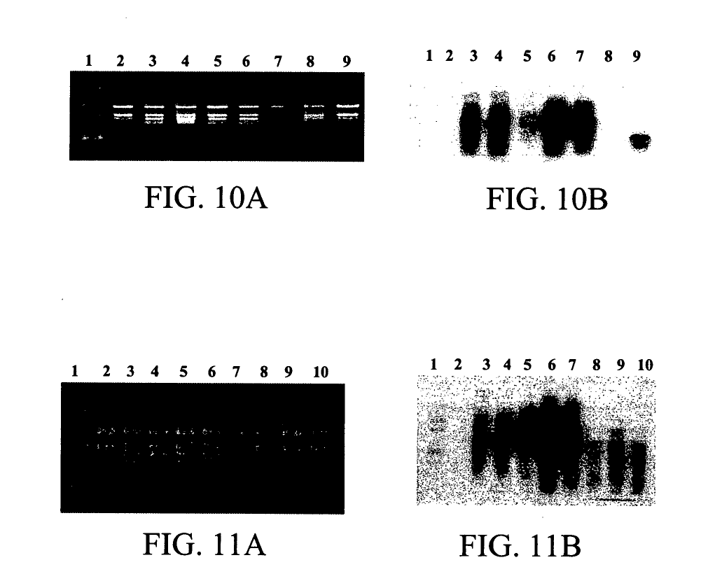

FIGS. 10A and 10B. Northern blot analysis of light chain transcripts in the transgenic lines transformed with the pZS-TP-Guy's 13 and pLD-TP Guy's 13 vectors. FIG. 10A. RNA gel before transfer. FIG. 10B. RNA blot probed with radiolabelled light chain DNA probe. Lane 1, RNA ladder; Lane 2, control untransformed Petit Havana; Lanes 3-5, transgenic lines transformed with pZS-TP-Guy's 13; Lanes 6 and 7, transgenic lines transformed with pLD-TP-Guy's 13; Lane 8, post-transcriptionally silenced nuclear transformant CAR8841; Lane [nine] 9, expressing nuclear transformant CAR517.

FIGS. 11A and 11B. Northern Blot analysis of heavy chain transcripts in the transgenic lines transformed with the pZS-TP-Guy's 13 and pLD-TP Guy's 13 vectors. FIG. 11A. RNA gel before transfer. FIG. 11B. RNA blot probed with radiolabelled heavy chain DNA probe. Lane 1, RNA ladder; Lane 2, control untransformed Petit Havana; Lanes 3-5, transgenic lines transformed with pZS-TP-Guy's 13; Lanes 6 and 7, transgenic lines transformed with pLD-TP-Guy's 13; Lane 8, post-transcriptionally silenced nuclear transformant CAR8841; Lane 9, expressing nuclear transformant CAR517; Lane 10, expressing nuclear transform ant CAR532.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to describe more fully the state of the art to which this invention pertains.

### DEFINITIONS

The practice of the present invention will employ, unless otherwise indicated, conventional techniques of immunology, molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.); PCR 2: A PRACTICAL APPROACH (M. J. MacPherson, B. D. Hams and G. R. Taylor eds. (1995)); Harlow and Lane, eds (1988) ANTIBODIES: A LABORATORY MANUAL, and METHODS IN MOLECULAR BIOLOGY vol. 49, “PLANT GENE TRANSFER AND EXPRESSION PROTOCOLS,” H. Jones (1995).

As used in the specification and claims, the singular form “a,” “an,” and “the” include plural references unless the context clearly dictates otherwise. For example, the term “a cell” includes a plurality of cells, including mixtures thereof.

A “variable region” of an antibody refers to the variable region of the antibody's light chain or the variable region of the heavy chain either alone or in combination.

As used herein, a “polynucleotide” is a polymeric form of nucleotides of any length which contain deoxyribonucleotides, ribonucleotides, and/or their analogs. The terms “polynucleotide” and “nucleotide” as used herein as used interchangeably. Polynucleotides may have any three-dimensional structure and may perform any function, known or unknown. The term “polynucleotide” includes double-, single-stranded, and triple-helical molecules. Unless otherwise specified or required, any embodiment of the invention described herein that is a polynucleotide encompasses both the double-stranded form and each of two complementary single-stranded forms known or predicted to make up the double stranded form.

The term “polypeptide” is used in its broadest sense to refer to a compound of two or more subunit amino acids. The subunits may be linked by peptide bonds. As used herein the term “amino acid” refers to natural and/or unnatural or synthetic amino acids, including glycine and both the D and L optical isomers. A peptide of three or more amino acids is commonly called an oligopeptide if the peptide chain is short. If the peptide chain is long, the peptide is commonly called a polypeptide or a protein.

A “multimeric protein” as used herein refers to a globular protein containing more than one separate polypeptide or protein chain associated with each other to form a single globular protein in vitro or in vivo. The multimeric protein may consist of more than one polypeptide of the same kind to form a homodimeric or homotrimeric protein; the multimeric protein may also be composed of more than one polypeptide having distinct sequences to form, e.g., a heterodimer or a heterotrimer. Non-limiting examples of multimeric proteins include immunoglobulin molecules, receptor dimer complexes, trimeric G-proteins, and any enzyme complexes.

An “immunoglobulin molecule” or “antibody” is a polypeptide or multimeric protein containing the immunologically active portions of an immunoglobulin heavy chain and immunoglobulin light chain covalently coupled together and capable of specifically combining with antigen. The immunoglobulins or antibody molecules are a large family of molecules that include several types of molecules such as IgD, IgQ, IgA, secretory IgA (SIgA), IgM, and IgE. The term “immunoglobulin molecule” includes for example hybrid antibodies or altered antibodies and fragments thereof, including but not limited to Fab fragment(s) and single-chain variable fragments (ScFv).

An “Fab fragment” of an immunoglobulin molecule is a multimeric protein consisting of the portion of an immunoglobulin molecule containing the immunologically active portions of an immunoglobulin heavy chain and an immunoglobulin light chain covalently coupled together and capable of specifically combining with an antigen. Fab fragments can be prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using methods disclosed herein or any other methods known in the art.

An “ScFv fragment” of an immunoglobulin molecule is a protein consisting of the immunologically active portions of an immunoglobulin heavy chain variable region and an immunoglobulin light chain variable region covalently coupled together and capable of specifically combining with an antigen. ScFv fragments are typically prepared by expressing a suitable host cell the desired portions of immunoglobulin heavy chain variable region and immunoglobulin light chain variable region using methods described herein and/or other methods known to artisans in the field.

“Secretory component” is a fragment of an immunoglobulin molecule comprising secretory IgA as defined in U.S. Pat. No. 5,202,422 and U.S. Pat. No. 5,959,177, incorporated here by reference.

“J chain” is a polypeptide that is involved in the polymerization of immunoglobulins and transport of polymerized immunoglobulins through epithelial cells. J chain is found in pentameric IgM and dimeric IgA and typically attached via disulfide bonds.

A “protection protein” is a fragment of an immunoglobulin molecule comprising secretory IgA as defined in U.S. Pat. No. 6,046,037, incorporated herein by reference.

“Heterologous” means derived from a genotypically distinct entity from that of the rest of the entity to which it is compared. For example, a polynucleotide introduced by genetic engineering techniques into a different cell is a heterologous polynucleotide (and, when expressed, can encode a heterologous polypeptide). In particular, the term “heterologous” as applied to a multimeric protein means that the multimer is expressed in a host cell that is genotypically distinct from the host cell in which the multimer is normally expressed. For example, the exemplified human IgA multimeric protein is heterologous to a plant cell.

The term “immunologically active,” as used herein, refers to an immunoglobulin molecule having structural, regulatory, or biochemical functions of a naturally occurring molecule expressed in its native host cell. For instance, an immunologically active immunoglobulin produced in a plant cell by the methods of this invention has the structural characteristics of the naturally occurring molecule, and/or exhibits antigen binding specificity of the naturally occurring antibody that is present in the host cell in which the molecule is normally expressed.

A “gene” refers to a polynucleotide containing at least one open reading frame that is capable of encoding a particular protein after being transcribed and translated.

As used herein, “expression” refers to the process by which polynucleotides are transcribed into mRNA and/or the process by which the transcribed mRNA is subsequently translated into polypeptides or proteins.

The term “construct” or “vector” refers to an artificially assembled DNA segment to be transferred into a target plant tissue or cell. Typically, the construct will include the gene or genes of a particular interest, a marker gene and appropriate control sequences. The term “plasmid” refers to an autonomous, self-replicating extrachromosomal DNA molecule. In a preferred embodiment, the plasmid constructs of the present invention contain sequences coding for heavy and light chains of an antibody. Plasmid constructs containing suitable regulatory elements are also referred to as “expression cassettes.” In a preferred embodiment, a plasmid construct can also contain a screening or selectable marker, for example an antibiotic resistance gene.

The term “selectable marker” is used to refer to a gene that encodes a product that allows the growth of transgenic tissue on a selective medium. Non-limiting examples of selectable markers include genes encoding for antibiotic resistance, e.g., ampicillin, kanamycin, or the like. Other selectable markers will be known to those of skill in the art.

A “glycosylation signal sequence” is a three-amino acid sequence within a polypeptide, of the sequence N-X-S/T, where N is asparagine, X is any amino acid (except proline), S is serine, and T is threonine. The presence of this amino acid sequence on secreted proteins normally results, within the endoplasmic reticulum, in the covalent attachment of a carbohydrate group to the asparagine residue.

A “primer” is a short polynucleotide, generally with a free 3′ OH group, that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, typically a thermally-stable DNA polymerase enzyme. Methods for PCR are well known in the art and taught for example in MacPherson, et al. PCR: A Practical Approach (IRL Press at Oxford University Press (1991)). All processes of producing replicate copies of a polynucleotide such as PCR or gene cloning are collectively referred to herein as “replication.”

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure three or more strands forming a multi-stranded complex, a single self-hybridizing strand, or any combination of these. A hybridization reaction may constitute a step in a more extensive process, such as the initiation of a PCR reaction or the enzymatic cleavage of a polynucleotide by a ribozyme.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide if hybridization can occur between one of the strands of the first polynucleotide and the second.

As used herein, “homologous recombination” refers to a process whereby two homologous double-stranded polynucleotides recombine to form a novel polynucleotide.

A “transgenic plant” refers to a genetically engineered plant or progeny of genetically engineered plants. The transgenic plant usually contains material from at least one unrelated organism, such as a virus, another plant or animal.

A “control” is an alternative subject or sample used in an experiment for comparison purpose. A control can be “positive” or “negative.” For example, where the purpose of the experiment is to determine the presence of an exogenously introduced plasmid or the expression of a polypeptide encoded by such plasmid in a plant transformant or its progenies, it is generally preferable to use a positive control (a plant or a sample from a plan, carrying such plasmid and/or expressing the encoded protein), and a negative control (a plant or a sample from a plant lacking the plasmid of interest and/or expression of the polypeptide encoded by the plasmid).

“Guy's 13” is a monoclonal antibody against the surface antigen I/II of Streptococcus mutans and is described in U.S. Pat. No. 5,518,721 and PCT/US95/16889 incorporated herein by reference.

The term “Humanized,” as used herein, refers to a construct in which coding sequences for heavy and light chain variable regions from a species other than human have been fused, via genetic engineering to the coding sequences of the respective constant regions of human heavy and light chains. It also refers to the resulting antibodies.

“Codon optimization” is the process of customizing a transgene so that it matches the bias of highly expressed genes in the genome in which it is to be expressed. For most amino acids there are two or more (up to six) different codons that can be used in mRNA. Every genome has a “bias” in the codons it uses, especially for highly expressed proteins. Changing the codon usage of a heterologous gene has been shown in many systems to increase the expression of that gene.

As used herein an “operative ligand” is a polypeptide sequence that functionally interacts with or binds to another protein, polypeptide, carbohydrates or nucleic acid for a preferred function. Non-limiting examples of an operative ligand would be ICAM-1, which binds to human rhinovirus, or an ScFv that binds to a particular epitope.

Usefulness of the Invention:

Treatment of disease with antibodies is known as passive immunotherapy. This is distinguished from active immunotherapy, where vaccination stimulates the body's own antibody response. The efficacy of passive immunotherapy has been demonstrated in treatment of a number of infectious diseases, in both animals and humans. A major impediment to the commercialization of many types of passive immunotherapy is the need for repetitive delivery of large amounts of antibody to the site of the disease to overcome rapid clearing of the antibodies from the body. The production of antibodies by traditional methods is much too expensive to be practical for many types of passive immunotherapy. This is why production in plastids is such an attractive alternative.

For topical, enteric and mucosal use, secretory IgA (SIgA) is the preferred antibody isotype. SIgA is the most abundant immunoglobulin found in the body and the most important form found in mucosal secretions, such as saliva, tears, breast milk and mucus of the bronchial, genitourinary, and digestive tracts (Kerr, 1990). It is composed of 10 polypeptides: 4 light chains, four IgA heavy chains, a J chain and a secretory component (SC), resulting in a total molecular weight of ˜400 kDa. Binding of SIgA to bacterial and viral surface antigens prevents attachment of pathogens to the mucosal cells, and, once attachment is blocked, viral infection and bacterial colonization is inhibited.

SIgA has demonstrated superiority over other antibodies for use in passive mucosal immunotherapy. It is more protease resistant than IgG or IgA, thus making it more stable in the gastrointestinal tract (Brown et al., 1970; Crottet and Corthesy, 1998, Renegar et al, 1998) and buccal mucosa (Ma et al., 1998). Recent work at Planet demonstrated that in the presence of pepsin at pH 2.5, antigen binding of an IgG antibody lasted 5 minutes versus 5 hours for the same antibody prepared as an SIGA plantibody. Such stability will be an important feature of antibodies used for the treatment of gastrointestinal tract infections, such as rotavirus and Clostridium difficile. SIgA has twice as many binding sites than IgG, thus giving it an additional advantage where avidity is important. The superiority of SIgA over IgG or IgA has been demonstrated in a number of studies: 1) SIgA protected mice against group A Streptococci, but serum did not, even though the IgG had a higher titer by ELISA and opsonized cells more effectively in a mouse model (Bessen and Fischetti., 1988); 2) Mice were protected against influenza virus by intravenous injection of polymeric IgA (which was transported into nasal secretions as SIgA) while IgG1 and monomeric IgA were ineffectual (Renegar and Parker, 1991); and 3) Anti gp160 SIgA blocked transcytosis of HIV in human cells better than IgG, despite having lower specific activity (Hocini et al., 1997).

Plastid Transformation Vectors:

Antibody expression in transgenic tobacco was accomplished using two plastid expression vectors pLD and pZS, as shown in FIGS. 1C and 2C. Both plastid vectors contain the 16S rRNA promoter (Prrn) driving the selectable marker gene aadA (aminoglycoside adenylyl transferase, conferring resistance to spectinomycin) followed by the psbA 3′ region (the terminator from a gene coding for photosystem II reaction center components) from the tobacco plastid genome. The only difference between these two plastid vectors is the site of integration of foreign genes into the plastid genome. The tobacco vector (pZS) integrates the aadA gene into the spacer region between rbcL (the gene for the large subunit of RuBisCo) and orf512 (the accD gene) of the tobacco plastid genome. This vector is useful for integrating foreign genes specifically into the tobacco plastid genome; this gene order is not conserved among other plant plastid genomes. On the other hand, the universal plastid expression/integration vector (pLD) uses trnA and trnI genes (plastid transfer RNAs coding for alanine and isoleucine), from the inverted repeat region of the tobacco plastid genome, as flanking sequences for homologous recombination. This vector can be used to transform plastid genomes of several other plant species (Daniell et al. 1998) because the flanking sequences are highly conserved among higher plants. Because the universal vector integrates foreign genes within the Inverted Repeat region of the plastid genome, it should double the copy number of antibody genes (from 5,000 to 10,000 copies per cell in tobacco). Furthermore, it has been demonstrated that homoplasmy is achieved even in the first round of selection in tobacco probably because of the presence of a plastid origin of replication within the flanking sequence in the universal vector (thereby providing more templates for integration). Because of these and several other reasons, foreign gene expression was shown to be much higher when the universal vector was used instead of the tobacco vector (Guda et al. 2000).

### EXAMPLES

The following examples are intended to illustrate, but not limit, the scope of the invention.

### Example #1

### An IgA Antibody Against a Bacterial Surface Protein Expressed in Plastids

A. Preparation of Antibody Heavy and Light Chain Expression Cassette

For the first antibody to be expressed in plastids, we chose to use the binding region of a murine Mab known as “Guy's 13” (discovered at Guy's Hospital, London), which recognizes the 185 kDa surface antigen of Streptococcus mutans, the bacteria that causes cavities (Smith and Lehner, 1989). Short-term passive immunotherapy with Guy's 13 was shown to eliminate these cariogenic bacteria for periods of up to two years (Ma and Lehner, 1990). The potential worldwide market for this one antibody may approach several billion dollars per year, and require antibody produced inexpensively and in large quantities. Planet Biotechnology scientists have recently constructed humanized versions of the Guy's 13 antibody for plant nuclear expression. The preferred heavy chain construct consists of the Guy's 13 heavy chain variable region fused to the human IgA2m(2) constant region. This heavy chain sub-isotype is resistant to the bacterial proteases that specifically target IgA1 (Kerr, 1990). The light chain construct is a fusion of the Guy's 13 kappa chain variable region and the human kappa constant region. Expression of these two immunoglobulin chains, along with human J chain and human SC have resulted in the assembly in transgenic tobacco of a humanized Guy's 13 SIGA plantibody, which we call CaroRx.

To prepare the humanized Guy's 13 heavy and light chain genes for plastid transformation, coding sequences were amplified, using PCR, from expression cassettes designed for nuclear expression. To facilitate sub-cloning, primers were engineered to incorporate a ribosome binding site utilized by the plastid protein translation machinery, and a methionine codon (in place of the signal peptides found in the nuclear expression constructs). H and L chain PCR products were individually cloned into the vector pCR-Script (Stratagene) and their sequences verified.

Both clones were cut with BamH I, creating cohesive ends at the 3′ end of the H chain and at the 3′ and 5′ ends of the L chain, resulting in excision of the L chain. The L chain fragment was ligated adjacent to the 3′ end of the H chain (with an intervening stop codon and ribosome biding site) yielding a vector, pCR-ScriptGuy's 13, that contained both, H and L chain fragments.

The sequence of the expression cassette between the two Xba I sites in pLD-TP-Guy's 13 is shown in SEQ ID NO. 1. Nucleotides 1-16 comprise linker sequences and a ribosome binding site. Nucleotides 17-1381 comprise a sequence encoding a mouse heavy chain variable/human IgA2m(2) constant hybrid with linker sequences. The native mouse signal peptide has been replaced with methionine (nt 17-19). The heavy chain variable region (nt 20-358) is from the murine monoclonal Guy's 13 (Smith and Lehner, 1989; U.S. Pat. Nos. 5,518,721 and 5,352,446, herein incorporated by reference). The sequence of the human IgA2m(2) constant region (nt 359-1381) has been previously published (Chintalacharuvu et al., 1994). Nucleotides 1382-1408 comprise stop codon, linker sequences and a ribosome binding site. Nucleotides 1409-2050 comprise a sequence encoding a mouse light chain variable/human kappa constant hybrid with linker sequences. The native mouse signal peptide has been replaced with methionine (nt 1409-1411). The light chain variable region (nt 1412-1731) is from the murine monoclonal Guy's 13 (Smith and Lehner; U.S. Pat. Nos. 5,518,721 and 5,352,446). The sequence of the human kappa constant region (nt 1732-2050) has been previously published (Hieter et al., 1980).

The pCR-ScriptGuy's 13 vector was digested with Xba I to excise the HI/L chain insert, and the insert was ligated with Xba I-digested and dephosphorylated pLD vector (Universal vector). The resulting plasmid was designated as pLD-TP-Guy's 13 (FIG. 1). The sequences encoded are chimeric, consisting of mature variable regions from Guy's 13 heavy and light chains fused to the constant regions of human IgA2m(2) heavy chain and kappa light chain. A separate sample of the pCR-ScriptGuy's 13 vector was digested with Spe I to excise the H/L chain insert, and the insert was ligated with Spe I-digested and dephosphorylated pZS vector (Tobacco vector; FIG. 2). The resulting plasmid was designated as pZS-TP-Guy's 13.

B. Expression of pLD-TP-Guy's 13 and pZS-TP-Guy's 13 in E. coli:

Since the transcriptional and translational machinery of the plastid is similar to the transcriptional and translational machinery of E. coli (Brixey et al., 1997), it is possible to check the expression of Guy's 13 construct in E. coli. The transcriptional efficiency of the 16S promoter is as good as the transcriptional efficiency of the T7 promoter in E. coli (Brixey et al., 1997, Guda et al., 2000). E. coli XLI Blue MRF TC cells were transformed with pLD-TP-Guy's 13 and pZS-TP-Guy's 13 vectors, and were selected on LB medium with ampicillin (100 μg/mL). Transformed colonies were tested for the presence of the correct coding sequence insert by plasmid isolation and restriction digestion.

In one set of experiments, E. coli cells were lysed in TBS buffer (20 mM Tris-HCl, pH 8, 150 mM NaCl) containing 2 mM PMSF by sonication. Lysates were boiled for 5 min with an equal volume of 2× sample buffer [3.55 mL deionized water, 1.25 mL 0.5 M Tris-HCl, pH 6.8, 2.5 mL glycerol, 2.0 mL 10% (w/v) SDS, 0.2 mL 0.55% (w/v) bromophenol blue] and electrophoresed on 12% polyacrylamide gels according to the standard procedure. In the other set of experiments, aliquots of cells were centrifuged in micro-centrifuge tubes at 14,000 rpm for 2 min and pellets were washed with TBS buffer. Pellets were re-suspended in equal volumes of TBS buffer containing 2 mM PMSF and 2× sample buffer, boiled for 5 min and electrophoresed on 12% polyacrylamide gels according to the standard procedure. The gels were blotted onto nitrocellulose membranes. The unoccupied binding sites on the blots were blocked by incubating them in blocking buffer [10 mM Tris-HCl, 0.5 M NaCl, 0.05% Twin 20 (v/v), and 5% non-fat dry milk (w/v)] at room temperature for 1 h. After blocking, blots were incubated with an appropriate antibody labeled with alkaline phosphatase at room temperature for 2 h. Blots were washed three times at room temperature in blocking buffer without non-fat dry milk. After washing, blots were developed using the Alkaline Phosphatase Conjugate Substrate Kit according to the manufacturer's instructions (Bio-Rad, Hercules, CA).

Results of Western blot analysis indicated that the Mᵣ of the heavy chain was approximately 55 kDa (FIG. 4). The Mᵣ of the light chain was approximately 26 kDa (FIG. 3). It was also noticed that the heavy chain protein tended to form aggregates with very low mobility on the gel, which were detected at the top, above the 200 kDa protein marker band. Aggregates of heavy and light chains were also confirmed by the presence of smears above the 55 and 26 kDa bands of heavy and light chain respectively.

C. Bombardment and Regeneration of Plastid Transgenic Plants:

After confirming the presence of the Guy's 13 insert in both vectors, and testing the constructs in E. coli, plasmid DNA was purified and used for bombardment. Tobacco (Nicotiana tabacum cv. Petit Havana) plants were grown aseptically by germination of seeds on MSO medium containing MS salts (4.3 g/liter), B5 vitamin mixture (myo-inositol, 100 mg/liter; thiamine-HCl, 10 mg/liter; nicotinic acid, 1 mg/liter; pyridoxine-HCl, 1 mg/liter), sucrose (30 g/liter) and phytagar (6 g/liter) at pH 5.8 (Ye et al., 1990). Fully expanded, dark green leaves of about two month old plants grown under sterile conditions were used for bombardment.

Leaves were placed abaxial side up on a Whatman No. 1 filter paper laying on RMOP medium (Daniell, 1993) in standard petri plates (100×15 mm) for bombardment. Tungsten (1 μm) or Gold (0.6 μm) microprojectiles were coated with plasmid DNA (plastid vectors) and bombardments were performed with the biolistic device PDS 1000/He (Bio-Rad) as described by Daniell (1997). Following bombardment, petri plates were sealed with Parafilm and incubated at 24° C. in the dark. Two days after bombardment, leaves were cut into small pieces of ˜5 mm² in size and placed on selection medium (RMOP containing 500 μg/mL of spectinomycin dihydrochloride) with the abaxial side touching the medium in deep (100×25 mm) petri plates (˜6 pieces per plate). The regenerated spectinomycin-resistant shoots were cut into small pieces (˜2 mm²) and subcloned into fresh deep petri plates (˜5 pieces per plate) containing the same selection medium. Resistant shoots resulting from this second round of selection were then tested for the presence of the Guy's 13 construct (integration) using PCR (see below) and only transgenic shoots were transferred to rooting medium (MSO medium supplemented with IBA, 1 mg/L and spectinomycin dihycrochloride, 500 mg/L). These plants are designated T0 plants. Rooted plants were transferred to soil and grown at 26° C. under continuous lighting conditions for further analysis (FIG. 5). Seed collected from T0 plants were germinated on specinomycin, and then transferred to soil. These plants are designated T1 plants.

Spectinomycin/streptomycin resistant clones were observed within 3-6 weeks after bombardment. Total DNA from unbombarded and transgenic plants was isolated using DNeasy Plant Mini Kit (Qiagen, Valencia, Calif.). PCR was performed in order to distinguish: a) true transformants from spontaneous mutants and b) plastid transformants from nuclear transformants. DNA was amplified using Taq PCT core kit (Qiagen, Valencia, Calif.), using standard protocols (Sambrook et al., 1989). Samples were amplified in the Perkin Elmer™ 92s GeneAmp PCR system 2400. PCR products were analyzed by electrophoresis on 0.8% agarose gels.

For T0 plants transformed by pLD-TP-Guy's 13, two primers (3P and 3M) were used to confirm integration of the spectinomycin resistance gene (aad A) into the proper location in the plastid (to distinguish transformants from mutants). Primer 3P anneals to the 16S rRNA gene and primer 3M binds to the aadA coding region (FIG. 1C). The 3P primer anneals only with the plastid genome, so no PCR product can be obtained with nuclear transgenic plants. FIG. 1A shows that the expected size PCR product (1.65 kb) was obtained with the 3P and 3M primers, confirming integration of foreign genes into the plastid genome. To determine that the gene(s) of interest (antibody H and L genes) have been integrated without rearrangement, primers 5P and 2M were used. One primer anneals to the aadA coding sequence and the other anneals to the trnA region to confirm integration of the entire gene cassette (FIG. 1C). The presence of the expected size PCR product (3.6 kb, FIG. 1B) confirmed that the entire gene cassette was integrated and that there were no internal deletions or loop outs during integration via homologous recombination.

For T0 plants transformed by the 13pZS-TP-Guy's 13 vector, two primers were used in order to test the integration event (i.e., to distinguish transformants from mutants). One primer (7P) anneals to the rbcL 3′ region and the other (8M) anneals to the aadA gene to test integration of the aadA gene in transgenic plants (FIG. 2C). FIG. 2B shows that the expected size PCR product (0.9 kb) was obtained with this primer pair, confirming integration of foreign genes into the genome. No PCR product was obtained with specintomycin-resistant mutant plants using this set of primers. In order to test integration of genes into the plastid genome, two primers were used. One primer (8P) anneals to the rbcL 5′ gene while another anneals to the aadA gene (8M). Because the rbcL 5′ primer anneals only with the plastid genome, no PCR product was obtained with nuclear transgenic plants and mutant plants using this set of primers. The presence of the expected size PCR product (2.1 kb) confirmed plastid integration of both foreign genes (FIG. 2A). Plastid transgenic plants containing the antibody H and L chain genes were subjected to a second round of selection in order to achieve homoplasmy.

D. Southern Blot Analysis:

Southern blotting was used to test homoplasmy. That is, it establishes that the transformed genome (with antibody genes inserted) is the only one present. Total DNA was extracted from leaves of transformed and wild-type (control) plants using the DNeasy Plant Kit (Qiagen Inc.). Total DNA was digested with Bgl II, electrophoresed on 0.7% agarose gels and transferred to Duralon-UV membranes (Stratagene, CA). A 1.8 kb Bgl II/EcoR V fragment containing flanking sequences of the pZS vector was used as a probe for the lines transformed with the pZS-TP-Guy's13 vector (FIG. 8). A 0.81 kb Bgl II/BamH I fragment containing flanking sequences of the pLD vector was used as a probe for the lines transformed with the pLD-TP-Guy's 13 vector (FIG. 9). The probes were labeled with ³²P-dCTP using the Ready To Go kit (Pharmacia Biotech, NJ). The blots were prehybridized using Quickhyb prehybridization solution (Stratagene, CA). The blots were hybridized and washed according to the manufacturer's instructions.

The native size fragment present in the non-transformed control should be absent in the transgenics. The presence of a large fragment (due to insertion of foreign genes within the flanking sequences) and absence of the native small fragment establishes the homoplasmic nature of our transformants (Daniell et al., 1998; Kota et al., 1999; Guda et al., 2000). In the case of T0 lines transformed with the pLD-TP-Guy's 13 vector, 4.47 kb and 7.87 kb bands were observed (FIG. 9, lanes 4-6). In the case of control (untransformed) Petit Havana, only the 4.47 kb band was observed (FIG. 9, lane C). In the case of TO lines transformed with the pZS-TP-Guy's 13 vector, 2.6 kb and 6.0 kb bands were observed (FIG. 8, lanes 4-6). In case of control (untransformed) Petit Havana only the 2.6 kb band was observed. In the case of T I lines of both kinds, the wild-type bands (4.47 for the pLD and 2.6 for the pZS transformants) were either absent or very faint (FIGS. 9 and 8, lanes 1-3).

E. Northern Blot Analysis:

Northern blots were performed to test the efficiency of transcription of the antibody genes. Total RNA was isolated from 150 mg of frozen leaves of transformed and untransformed plants using the “Rneasy Plant total RNA Isolation Kit” (Qiagen Inc., Chatsworth, Calif.). RNA (9 μg of all samples except #8841, which had 6.5 μg) was denatured by formaldehyde treatment, separated on a 1.2% agarose MOPS gel in the presence of formaldehyde and transferred to Duralon-UV membranes (Stratagene, CA). Probe DNAs (antibody H and L chain coding regions) were labeled with ³²P-dCTP using the Ready To Go kit (Pharmacia Biotech, NJ). The blots were prehybridized using Qiuckhyb prehybridization solution (Stratagene, CA). The blots were hybridized and washed according to the instructional manual (Stratagene, CA). The transcript levels were quantified using the Storm 840 phosphoimager system (Molecular Dymanics).

Abundant transcripts that hybridized to both light chain and heavy chain probes were detected in RNA from plastid transformants (FIGS. 9 and 10). These transcripts were larger in size than transcripts detected in nuclear transgenic plants, consistent with the presence of polycistronic transcripts in the transgenic plastids. The transcription levels between the nuclear transformants and plastids transformants were compared. The transcription levels between the plastid transformant lines transformed with the pZS-TP-Guy's 13 vector and the lines transformed with the pLD-TP-Guy's 13 vector were also compared. The plastid transformants transformed with the pLD-TP-Guy's 13 vector expressed 13/24 fold more transcripts. The plastid transformants transformed with the pLD-TP-Guy's 13 vector expressed two fold more transcripts than the plastid transformants transformed with the pZS-TP-Guy's 13.

F. Western Blot Analysis:

Two methods were used to extract proteins from the plastids. In the first method, plant leaves (100 mg) were ground in liquid nitrogen and resuspended in 150 μl of TBS buffer buffer (20 mM Tris-HCl, pH 8, 150 mM NaCl). Samples were mixed well by vortexing. Equal volumes of the plant extracts and 2×SDS sample buffer [10 mM TRIS-Cl, 4% SDS, 1 mM (Na)₂EDTA, 15% glycerol (v/v) and 0.05% bromophenol blue (w/v)] were mixed, boiled for 4 minutes, briefly centrifuged, and the supernatant loaded on polyacrylamide gels. In the second method the plant leaves (100 mg) were directly ground in 2×SDS sample buffer, boiled for 4 min, briefly spun and loaded on polyacrylamide gels. Samples treated with reductant were electorphoresed on 12% acrylamide gels. Non-reduced samples were electrophoresed on 7% acrylamide gels. The gels were electro-blotted onto nitrocellulose membranes in a Trans-Blot Electrophoreic transfer cell (BioRad, CA) following the manufacturer's instructions. The unoccupied binding sites on the blots were blocked by incubating them in blocking buffer [10 mM Tris-HCl, 0.5 M NaCl, 0.05% Tween 20 (v/v), and 5% non-fat dry milk (w/v)] at room temperature for 1 h. After blocking, blots were incubated for 2 hours at room temperature with alkaline phosphatase-conjugated goat anti-human IgA or goat anti-human kappa antibody, diluted 1:2000 in blocking buffer. Blots were washed three times at room temperature in TBS. After washing, blots were developed using the Alkaline Phosphatase Conjugate Substrate Kit (Bio-Rad, Hercules, CA) according to the manufacturer's instructions.

Bands of approximately 26 Mr were detected using the alkaline phosphate (AP) conjugated goat anti human kappa antibody from the samples that were electrophoresed under reducing conditions. Bands of approximately 55 Mr were detected using the AP conjugated goat anti human IgA antibody from the samples that were elecrophoresed under reducing conditions (FIG. 6). Bands of approximately 180 Mr were detected using the AP conjugated goat anti human kappa antibody from the samples that were electrophoresed under non reducing conditions (FIG. 7). This was considered evidence of expression of both heavy and light chains, and assembly into an immunoglobulin.

G. ELISA Assays of Antibody Assembly

Determination of antibody concentration and detection of antibody binding function is performed by ELISA. Assays are done on crude extracts of leaves made by homogenizing small samples in two volumes of extraction buffer (25 mM Tris pH 7.5, 150 mM NaCl, 10 mM EDTA, 1% sodium citrate, 1% PVPP, 0.2% sodium thiosulfate). Homogenates are centrifuged in microfuge tubes for 10 minutes to pellet plastids and assays performed in the lysed supernatant.

The concentration of assembled antibody is determined using a double antibody sandwich ELISA. In this assay, an antibody against kappa chain bound to the plate captures any plantibody in the extract, which is detected by antibody against IgA heavy chain (to detect assembled IgA or SIgA), or by antibody against secretory component (to detect assembled SIgA). Microtiter wells are coated overnight at 4° C. with goat anti-human light chain-specific antibodies (50 μl/well at 4 μg/mL in PBS). Plates are washed, then blocked with PBS+5% non-fat dry milk 1 hour at room temperature. Supernatant is added to the microtiter plate in serial twofold dilutions (in PSB+5% non-fat dry milk) and the plate is incubated 1 hour at 37° C. Wells are washed, then incubated for 1 h at 37° C. with the appropriate goat anti-human chain-specific antibodies conjugated with horseradish peroxidase (Fisher Scientific), diluted 1:2000 in PSB+5% non-fat dry milk. For plants produced in the first phase of work (transformed only with heavy and light chains) the detecting antibody is anti-human IgA HRP. For plants transformed with all the components of SIgA the detecting antibody is anti-human secretory component-HRP (secretory component will not assemble onto an antibody without J chain). Plates are washed with water, and antibody complexes are detected by adding HRP substrate [0.1 M sodium citrate, pH 4.4 containing 0.0125% hydrogen peroxide and 0.40 mg/mL 2.2′-azino-bis (3-Ethylbenzthiazoline-6-sulfonic acid)], and incubating 30 minutes at room temperature. Color development (absorbance at 405 nm) is determined using a Benchmark Microplate Reader (Bio-Rad). Antibody concentrations in μg/mL) are determined by comparison with standard curve of human SIgA (Sigma), using a four-parameter logistic fit (SigmaPlot 3.0).

H. ELISA Assay of Antibody Binding Function:

The ability of plastid-produced antibody to bind to the cognate antigen, Streptococcal antigen I/II (SAI/II), is determined using ELISA. SA I/II is purified from culture supernatants of Steptococcus mutans strain IB 162 by the method of Russell et al. (1980). Microtiter plates are coated with purified SA I/II (50 μL/well at 2 μg/mL) overnight at 4° C. Plates are washed, blocked with PBS+5% nonfat dry milk, and probed 1 hr at 37° C. with a dilution series of plant extract. Bound antibodies are detected using the appropriate HRP-conjugated goat anti-human second antibody, and the plates processed exactly as described above for the double-antibody sandwich ELISA. A reference standard lot of Guy's 13 SIgA (produced by nuclear transgenic plants) is always tested along with test samples to control for assay to assay variation. Binding titer is calculated as the dilution of test antibody (normalized to 1 mg/mL as determined by the double antibody sandwich ELISA) necessary to generate an ELISA signal that is 50% of the maximum signal.

I. Purification of Antibody:

Plastids are first isolated from a crude homogenate of leaves by a simple centrifugation step at 1500×g. This eliminates most of the cellular organelles and proteins (Daniell et al., 1983, 1986). Then plastids are burst open by re-suspending them in a hypotonic buffer (osmotic shock). This is a significant advantage because there are fewer soluble proteins inside plastids when compared to hundreds of soluble proteins in the cytosol. The homogenate is centrifuged at 10,000 g for 10 minutes (4° C.) and the pellet discarded. Purification of antibody is performed as described in Ma et al. (1998), with some modification. Plastid homogenate is mixed with two volumes of extraction buffer (25 mM Tris pH 7.5, 150 mM NaCl, 10 mM EDTA, 1% sodium citrate, 1% PVPP, 0.2% sodium thiosulfate). The mixture is centrifuged at 17,000 g for 60 min, and the supernatant filtered through a 0.2 μM nominal cut-off filter. Filtrate is concentrated by diafiltration using a 300-kD MWCO tangential flow cassette (Millipore Corporation). Immunoglobulins are precipitated with 40% ammonium sulfate, collected by centrifugation at 17,000 g for 15 min, and then re-suspended in phosphate buffered saline (PBS).

J. Inheritance of Introduced Foreign Genes:

Some of the initial tobacco transformants are allowed to self-pollinate, whereas others are used in reciprocal crosses with control tobacco plants (transgenics as female acceptors and pollen donors; testing for maternal inheritance). Harvested seeds (T1) are germinated on media containing spectinomycin or other appropriate selective agents. Achievement of homoplasmy and mode of inheritance can be classified by observing germination results. Homoplasmy is indicated by totally green seedlings (Daniell et al., 1998) while heteroplasmy is displayed by variegated leaves (lack of pigmentation, Svab and Maliga, 1993). Lack of variation in chlorophyll pigmentation among progeny underscore the absence of position effect, an artifact of nuclear transformation. Maternal inheritance is demonstrated by sole transmission of introduced genes via seed generated on transgenic plants, regardless of pollen source (green seedlings on selective media). When transgenic pollen is used for pollination of control plants, resultant progeny do not contain resistance to chemical in selective media (appear bleached; Svab and Maliga, 1993). Molecular analyses (PCR, Southern, and Northern) confirm transmission and expression of introduced genes, and T2 seed is generated from those confirmed plants.

### Example #2

### Optimizing the Codon Usage of Antibody Genes to Maximize Expression in Plastids

Codon optimization has been used previously to successfully increase the level of transgenic protein in plants (McBride et al., 1995; Rouwendal et al., 1997 Horvath et al., 2000). In the case of a β-(1,3-1,4)-glucanase expressed in barley, codon optimization resulted in at least a 50-fold increase in expression (Horvath et al., 2000). Two factors contribute to codon bias in all organisms. One is the overall composition of the genome, which contributes to a bias in degenerate positions of codons (Bernardi et al., 1986). In tobacco plastid non-coding regions, the AT content is 69.6%. An AT-rich cry1A gene (encoding a Bacillus thuringiensis toxin) accumulated to much higher levels in plastids than the same gene having nuclear codon preferences (McBride et al., 1995). High AT content, however, is not the whole story. The second factor is selection for translation efficiency, resulting in a bias for specific codons (Ikemura et al., 1985). It has been proposed (Morton, 1993; Morton, 1998) that codon use in plastids is adapted to tRNA levels and that highly expressed genes have a greater bias in codon use as a result of selection for increased translation efficiency. Modification of a transgene to match the codon usage of highly expressed genes should result in even higher levels of expression. We devised a codon optimization table (Table 2) based on published observations of codon useage in plastids (Morton, 1993; Morton, 1998; Morton and So 2000). Essentially, we hypothesized that any gene utilizing the codons found in this table, and utilizing the rules listed below, would express at a higher level in plastids than the native gene.
Rule #1: The primary codon is used, unless conditions met in rules number 2 and 3 are present.
Rule #2: If a codon ending with C is followed by a codon beginning with G, the secondary codon is used, so as to avoid the combination NNC GNN, in which N represents any nucleotide and NNC and GNN are adjacent codons.
Rule #3: If the same amino acid is encoded twice with four or fewer intervening amino acids (for example, LXXXL, where L is Leucine and X is any amino acid) the secondary codon is used to encode one of the amino acids (either the first or second L, in the example), being careful to avoid violating Rule #2.
Rule #3: If the same amino acid is encoded three times with four or fewer intervening amino acids between the first and third occurence (for example, LLXXL, where L is Leucine and X is any amino acid) the tertiary codon is used to encode one of the amino acids (either the first or second L, in the example), being careful to avoid violating Rule #2.

Rule #4: If using the primary codon would result in significant secondary RNA structure (such as a stable stem-loop), the secondary codon is used.

TABLE 2
Optimal Codons for Plastid Expression

| Amino | Primary | Secondary | Tertiary |
|---|---|---|---|
| Acid | Codon | Codon | Codon |
| Leu | TTA | CTT | TTG |
| Ser | TCT | AGC | AGT |
| Arg | CGT | AGA | CGC |
| Pro | CCT | CCA | |
| Thr | ACT | ACC | |
| Val | GTA | GTT | |
| Ala | GCT | GCA | |
| Gly | GGT | GGA | |
| Ile | ATr | ATC | |
| His | CAC | CAT | |
| Gln | CAA | CAG | |
| Glu | GAA | GAG | |
| Asp | GAT | GAC | |
| Asn | AAC | AAT | |
| Lys | AAA | AAG | |
| Tyr | TAC | TAT | |
| Cys | TGT | TGC | |
| Phe | TTC | TTT | |

A synthetic gene was constructed that encoded a polypeptide consisting of the variable region of a murine anti-rotavirus monoclonal antibody fused to the constant region of human IgA2m(2) heavy chaing (Chintalacharuvu et al., 1994). The sequence of this chimeric gene was modified from the native mammalian gene sequences by codon optimization for plastid expression, using the rules in table 2. In addition, TAA was used as a stop codon. Synthesis of the gene was contracted to Entelechon GmbH. The gene was synthesized using the overlap extension PCR method (Rouwendal et al., 1997), but could be synthesized by various methods known to those skilled in the art. Another gene, encoding a polypeptide consisting of the variable region of a murine anti-rotavirus monoclonal antibody fused to the constant region of human kappa chain was synthesized by the same method, with codons optimized for plastid expression. Both synthetic genes were cloned into the vector pCR4TOPO (Invitrogen).

The plasmid containing the heavy chain sequence was cut with Sal I, and the plasmid containing the light chain sequence was cut with Sal I and Xho I. A Sal I/Xho I fragment containing the light chain sequence was then isolated and cloned into the Sal I site of the plasmid containing the heavy chain. The resulting bacterial clones were screened for a clone with the correct orientation (heavy chain followed by light chain with coding sequences in the same orientation). The heavy and light chain genes, with associated ribosome binding sites were then cut out together using Not I and Xba I, and cloned into the pLD vector.

The pLD vector with codon-optimized heavy and light chain coding sequences was used to transform tobacco plastids as described in Example 1. Transgenic plants are isolated and shown to contain high levels of human IgA.

### Example #3

### Expression of SIGA in Plastids with all Genes on one Vector

Expression of SIgA in plastids is accomplished by the simultaneous integration of four genes, IgA heavy chain, light chain, J chain and secretory component. These genes are expressed on a polycistronic message. A plasmid, based on pLD, is constructed containing the Guy's 13 heavy and light chains, and the mature-peptide coding regions of human J chain and SC genes, all downstream of the aadA gene and each having a ribosome binding site. The total size of this mRNA is over 4500 nt. Tobacco leaves are transformed by particle bombardment and transplastomic plants are selected by regeneration on antibiotic-containing medium by methods similar to those disclosed in Example #1. Appropriate primers are used for PCR analysis. Expression of J chain and SC is evaluated by western blotting, using antisera specific for human J chain and human secretory component. Detection of a band at ˜370 kDa with anti-IgA, anti-kappa, anti-J and anti-SC antibodies is considered evidence of assembled SIgA.

### Example #4

### Expression of SIGA in Plastids with J Chain and Secretory Component Genes on One Vector and Heavy and Light Chain Genes on Another Vector

Two plastid expression vectors, one containing heavy and light chain genes, and the other containing the J chain and secretory component genes are constructed by methods similar to those described in Example #1. The amino acid sequence of the J chain and secretory component encoded in the second vector are those described in U.S. Pat. No. 5,959,177 and U.S. Pat. No. 6,046,037, incorporated herein by reference. The two vectors use different plastid DNA flanking sequences, so that they integrate into the plastid chromosome in different locations. Tobacco leaves are transformed by particle bombardment and transplastomic plants are selected by regeneration on antibiotic-containing medium by methods similar to those disclosed in Example # 1. Appropriate primers are used for PCR analysis. Expression of J chain and SC is evaluated by western blotting, using antisera specific for human J chain and human secretory component. Detection of a band at ˜370 kDa with anti-IgA, anti-kappa, anti-J and anti-SC antibodies is considered evidence of assembled SIgA.

### Example #5

### Expression of a Chimeric Heavy Chain in Plastids

A fragment containing all 5 extracellular Ig-like domains of ICAM-1 is amplified from plasmid pIgAD5 (a gift of T. Springer) using the primers:

(SEQ ID NO.3)

5′-AAAATCTAGAGGAGGGATTTATGCAGACATCTGTGTCCCCCTCAAAA
GTC-3′
and

(SEQ ID NO.4)

5′-CATACCGGGGACTAGTCACATTCACGGTCACCTCGCG-3′

SEQ ID NO.3

5[′]′ AAAATCTAGAGGAGGGATTTATGCAGACATCTGTGTCCCCCTCA
AAAGTC-3[′]′
and

SEQ ID NO.4

5[′]′-CATACCGGGGACTAGTCACATTCACGGTCACCTCGCG-3[′]′

The resulting PCR product incorporates a ribosome-binding site utilized by the plastid protein translation machinery and a methionine codon upstream of the first amino acid of ICAM-1 . The PCR product is cut with Xba I and Spe I (underlined sequences) and cloned into a vector containing the human IgA2m(2) heavy chain constant region. The resulting chimeric gene encodes one continuous protein consisting of 5 domains of ICAM-1 and the constant region of IgA2m(2). The mature protein produced from this construct starts with the sequence of Met-Gln-Thr-Ser-Val- (SEQ ID NO. 5), and ends with the sequence -Lys-Asp-Glu-Leu (SEQ ID NO. 6). It is predicted to have 800 amino acids and a molecular weight of approximately 80,000. The sequence of the ICAM gene has been published (Staunton et al. 1988), and is incorporated herein by reference. The entire coding sequence of the chimeric gene is cut out with Xba I and cloned into the pLD vector. The resulting expression vector is used to transform tobacco plastids. The chimeric ICAM-1/IgA protein is expressed in transgenic plastids, and assembles into dimers. This multimeric protein comprises an immunoglobulin heavy chain fused to a functional ligand (ICAM-1 domains 1-5), and binds to a site on human rhinoviruses. It is used in a therapeutic manner to prevent rhinovirus colds.

### REFERENCES CITED

1. Artsaenko 0, Peisker M, zur Nieden U, Fiedler U, Weiler E W, Muntz K, Conrad U (1995) Expression of a single-chain Fv antibody against abscisic acid creates a wilty phenotype in transgenic tobacco. The Plant Journal 8:745-750.
2. Bernardi, G (1986) Compositional constraints and genome evolution. J Mol Evol 24; 1-11
3. Bessen D, Fischetti V A (1988) Passive acquired mucosal immunity to group A streptococci by secretory immunoglobulin A. J. Exp. Med. 167:1945-1950.
4. Boss M A, Kenten J H, Wood C R, Emtage J S (1984) Assembly of functional antibodies from immunoglobulin heavy and light chains synthesized in E. Coli. Nucleic acids research 12(9): 3791-3806.
5. Brixey J, Guda C, Daniell H (1997) The chloroplast psbA promoter is more efficient in E. coli than the T7 promoter for hyper expression of a foreign protein. Biotechnology Letters 19:395-400.
6. Brown W B, Newcombe R W, Ishizaka K (1970) Proteolytic degradation of exocrine and serum immunoglobulins. Journal of Clinical Investigation 49: 1374-1380.
7. Carlson P S (1973) The use of protoplasts for genetic research. Proc. Natl. Acad. Sci. USA 70:598-602.
8. Chintalacharuvu K R, Raines M, and Morrison S L (1994) Divergence of human alpha-chain constant region gene sequences. A novel recombinant alpha 2 gene. Journal of Immunology, 152:5299-5304.
9. Crottet P, Corthesy B (1998) Secretory component delays the conversion of secretory IgA into antigen-binding competent F(ab′)2: a possible implication for mucosal defense. J Immunol 161: 5445-5453.
10. Daniell H (1993) Foreign gene expression in chloroplasts mediated by tungsten particle bombardment. Methods Enzymol 217: 536-556.
11. Daniell H (1999 A) Genetically modified food crops: current concerns and solutions for the next generation crops. Biotechnology and Genetic Engineering Reviews 17:327-347.
12. Daniell H (1999 B) Environmentally friendly approaches to genetic engineering. In vitro Cellular and Developmental Biology-Plant, 35, 361-368.
13. Daniell H (1999 C) New tools for chloroplast genetic engineering. Nature Biotechnology 17, 855-856.
14. Daniell H (1999 D) GM crops: public perception and scientific solutions. Trends in Plant Science, 4:467-469.
15. Daniell H (1999 D) Universal chloroplast integration and expression vectors, transformed plants and products thereof, World Intellectual Property Organization WO 99/10513.
16. Daniell H, Datta R, Varma S, Gray S, Lee S B (1998) Containment of herbicide resistance through genetic engineering of the chloroplast genome. Nature Biotechnology 16: 345-348.
17. Daniell H, Krishnan M, McFadden B A (1991) Expression of B-glucuronidase gene in different cellular compartments following biolistic delivery of foreign DNA into wheat leaves and calli. Plant Cell Reports 9:615-619.
18. Daniell H, Krishnan M, Umabai U, Gnanam A (1986) An efficient and prolonged in vitro translational system from cucumber etioplasts. Biochem. Biophys. Res. Comun 135: 48-255.
19. Daniell H, McFadden B A (1988) Genetic Engineering of plant chloroplasts. U.S. Pat. Nos. 5,693,507; 5,932,479.
20. Daniell H, McFadden B A (1987) Uptake and expression of bacterial and cyanobacterial genes by isolated cucumber etioplasts. Proc Natl Acad Sci USA 84:6349-6353.
21. Daniell H, PoroboDessai A, Prakash C S, and Moar W J (1994) Engineering plants for stress tolerance via organelle genomes. In Biochemical and Cellular Mechanisms of Stress Tolerance in Plants, ed. J. H. Cherry, Springer-Verlag, Berlin NATO ASI Series Vol. H 86, 589-604.
22. Daniell H, Vivekananda J, Neilsen B, Ye G N, Tewari K K, Sanford J C (1990) Transient foreign gene expression in chloroplasts of cultured tobacco cells following biolistic delivery of chloroplast vectors. Proc Natl Acad Sci USA 87:88-92.
23. De Neve M, De Loose M, Jacobs A, Van Houdt H, Kaluza B, Weidle U, Depicker A (1993) Assembly of an antibody and its derived antibody fragment in Nicotiana and Arabidopsis. Transgenic Research 2: 227-237
24. De Neve M, De Buck S, De Wilde C, Van Houdt H, Strobbe I, Jacobs A, Van Montagu, Depicker A (1999) Gene silencing results in instability of antibody production in transgenic plants. Mol. Gen. Genet. 260: 582-592.
25. Drescher D F, Follman H, Haberlein I (1998) Sufitolysis and thioredoxin-dependent reduction reveal the presence of a structural disulfide bridge on spinach chloroplast fructose-1′6-biphosphatase. FEBS Letters. 424: 109-112.
26. Düring K, Hippe S, Kreuzaler F, Schell J (1990) Synthesis and self-assembly of a functional monoclonal antibody in transgenic Nicotiana tabacum. Plant Molecular Biology.15:281-293.
27. Ferri G, Comerio G, Iadarola P, Zapponi M C, Speranza M L (1978) Subunit structure and activity of glycedraldehyde-3-phosphate dehydrogenate from spinach chloroplasts. Biotic Biophys Acta 522:19-31.
28. Fiedler U, Phillips J, Artsaenko O, Conrad U (1997) Optimization of scFv antibody production in transgenic plants. Immunotechnology 3:205-216.
29. Fontes E B, Shank B B, Wrobel R L, Moose S P, OBrian G R, Wurtzel E T, Boston R S (1991) Characterization of an immunoglobulin binding protein homologue in the maize floury-2 endosperm mutant. Plant Cell 3, 483-496.
30. Gething M. J., Sambrook J (1992). Protein folding in the cell. Nature 355: 33-45.
31. Gould, F (1998) Sustainability of transgenic insecticidal cultivars:integrating pest genetics and ecology. Annu. Rev. Entomol. 43:701-726.
32. Guda, C., Lee, S. B., and Daniell, H. (2000). Stable expression of biodegradable protein based polymer in tobacco chloroplasts. Plant Cell Reports 19, 257-262.
33. Hiatt A, Cafferkey R, Bowdish K (1989) Production of antibodies in transgenic plants. Nature.342: 76-78.
34. Hieter P A, Max E E, Seidman J G, Maizel J V J, and Leder P. (1980). Cloned human and mouse kappa immunoglobulin constant and J region regions conserve homology in functional segments. Cell, 22:197-207.
35. Hocini H, Belec L, Iscaki S, Garin B, Pillot J, Becquart P, Bomsel M (1997) High-Level ability of secretory IgA to block HIV type I transcytosis: Contrasting secretory IgA and IgG responses to glycoprotein 160. HIV Research and Human Retroviruses, 23:1179-1184.
36. Horvath H, Huang J, Wong O, Kohl E, Okita T, Kannangara C G, von Wettstein D (2000) The production of recombinant proteins in transgenic barley grains. Proceedings of the National Acadamy of Sciences USA 97:1914-1919.
37. Hoyle B. (1999) Canadian farmers seek compensation for genetic pollution. Nat. Biotechnol. 17, 747-748.
38. Ikemura, T (1985) Codon usage and tRNA content in unicellular and multicellular organisms. Mol Biol Evol 2:13-35
39. Kerr M (1990) The structure and function of human IgA. Biochemical Journal, 271:285-296.
40. Kim J, Mayfield P S (1997) Protein disulfide isomerase as a regulator of chloroplast translational activation. Science 278:1954-1957.
41. Kota M, Daniell H, Varma S, Garczynski F, Gould F, Moar W J (1999) Overexpression of the Bacillus thuringiensis Cry2A protein in chloroplasts confers resistance to plants against susceptible and Bt-resistant insects. Proc. Natl. Acad. Sci. USA, 96:1840-1845.
42. Losey J E et al. (1999) Transgenic pollen harms monarch larvae, Nature 399, 214
43. Ma J K-C, Hiatt A, Hein M, Vine N D, Wang F, Stabila P, Van Dolleweerd C, Mostov K, Lehner T (1995) Generation and assembly of secretory antibodies in plants. Science. 268:716-719.
44. Ma J K-C, Hikmat B Y, Wycoff K, Vine N D, Chargelegue D, Yu L, Hein M B, Lehner T (1998) Characterization of a recombinant plant monoclonal secretory antibody and preventive immunotherapy in humans. Nature Medicine. 4 (5): 601-607.
45. Ma J K-C, Lehner T (1990) Prevention of colonization of Streptococcus mutans by topical application of monoclonal antibodies in human subjects. Archs. Oral. Biol. 35: 115S-122S.
46. Ma J K-C, Lehner T, Stabila P, Fux C I, Hiatt A (1994) Assembly of monoclonal antibodies with IgG I and IgA heavy chains in transgenic tobacco plants. Eur. J. Immunol.24: 131-138.
47. Ma J, Hikmat B, Wycoff K, Vine N, Chargelegue D, Yu L, Hein M, Lehner T (1998) Characterization of a recombinant plant monoclonal secretory antibody and preventive immunotherapy in humans. Nat. Med. 4: 601-606.
48. May G D, Mason H S, Lyons P C (1996) Application of transgenic plants as production systems for pharmaceuticals. In: ACS symposium series 647. Fuller et al eds., chapter 13, 194-204.
49. McBride K E, Svab Z, Schaaf D J, Hogen P S, Stalker D M, and Maliga P. 1995. Amplification of a chimeric Bacillus gene in chloroplasts leads to an extraordinary level of an insecticidal protein in tobacco. Bio/Technol., 13:362-365.
50. Melnick J., Aviel S., Argon Y. (1992). The endoplasmic reticulum stress protein GRP94, in addition to BiP, associates with unassembled immunoglobulin chains. J. Biol. Chem 267: 21303-21306.
51. Morton, B R (1993) Chloroplast DNA codon use: Evidence for selection at the psbA locus based on tRNA availability. Journal of Molecular Evolution 37:273-280.
52. Morton, B R (1998) Selection on the codon bias of chloroplast and cyanelle genes in different plant and algal lineages. Journal of Molecular Evolution 46:449-459.
53. Morton B R, and So B G (2000) Codon usage in plastid genes is correlated with context, position within the gene, and amino acid content. J Mol Evol 50:184-193.
54. Niller E, 1999. GM corn poses little threat to monarch. Nat Biotechnol, 17:1154.
55. Owen M, Gandecha A, Cockburn B, Whitelam G (1992; Synthesis of a functional anti-phytochrome single-chain Fv protein in transgenic tobacco. Bio/Technology. 10: 790-794.
56. Ranty B, Lorimer G, and Gutteridge S 1991. An intra-dimeric crosslink of large subunits of spinach ribulose-1,5-bisphosphate carboxylase/oxygenase is formed by oxidation of cysteine 247. Eur. J. Biochem, 200:353-358.
57. Renegar K B, Jackson G D, Mestecky J (1998) In vitro comparison of the biologic activities of monoclonal monomeric IgA, polymeric IgA, and secretory IgA. Journal of Immunology 160: 1219-23
58. Renegar K B, Parker A S J (1991) Passive transfer of local immunity to influenza virus infection by IgA antibody. J Immunol, 146:1972-1978.
59. Rogers S O, Bendich A J (1988) In: Gelvin S B, Schilperoot R A (eds) Plant molecular biology manual, Kluwer Academic Publishers, Dordrecht, Netherlands, pp. A6:1-10.
60. Rouwendal G J, Mendes O, Wolbert E J, Douwe de Boer A (1997) Enhanced expression in tobacco of the gene encoding green fluorescent protein by modification of its codon usage. Plant Molecular Biology 33:989-999.
61. Roy H (1989) Rubisco assembly: a model system for studying the mechanism of chaperonin action. Plant Cell. 1:1035-1042.
62. Ruelland E, Miginiac-Maslow M (1999) Regulation of chloroplast enzyme activities by thioredoxins: activation or relief from inhibition. Trends in Plant science 4: 136-141.
63. Russell M W, Bergmeier L A, Zanders E D, Lehner T (1980) Protein antigens of Streptococcus mutans: Purification and properties of a double antigen and its protease-resistant component. Infect. Immunity, 28:486-493.
64. Sambrook J, Fritsch E F, Maniatis T (1989) Molecular cloning. Cold Spring Harbor Press, Cold Spring Harbor, N.Y.
65. Schreuder H A, Knight S, Curmi P M, Andersson I, Cascio D, Sweet R M, Branden C I, Eisenberg D (1993) Crystal structure of activated tobacco rubisco complexed with the reaction-intermediate analogue 2-carboxy-arabinotol 1,5-bisphosphate. Protein Science. 2:1136-1146.
66. Scott S E and Wilkinson M J (1999) Low probability of chloroplast ovement from oilseed rape (Brassica napus) into wild Brassica rapa, Nat. Biotechnol. 17, 390-392.
67. Sidorov V A, Kasten D, Pang S Z, hajdukiewicz P T J, staub J M, Nehra, N S (1999) Stable chloroplast transformation in potato: use of green fluorescent protein as a plastid marker. Plant Journal 19: 209-216.
68. Skerra A, Pfitzinger I, Pluckthun A (1991) The functional expression of antibody Fv fragments in Escherichia coli: Improved vectors and a generally applicable purification technique. Bio/Technology 9: 273-278.
69. Skerra A, Pluckthun A (1988) Assembly of a functional immunoglobulin Fv fragment in Escherichia coli. Science 240:1038-1041.
70. Smith R, Lehner T (1989) Characterization of monoclonal antibodies to common protein epitopes on the cell surface of Streptococcus mutans and Streptococcus sobrinus. Oral Microbiol. Immunol. 4: 153-158.
71. Staunton D E, Marlin S D, Stratowa C, Dustin M L, Springer T A (1988). Primary structure of ICAM-1 demonstrates interaction between members of the immunoglobulin and integrin supergene families. Cell 52:925-933.
72. van Engelen F A, Schouten A, Molthoff J W, Roosien J, Salinas J, Dirkse W G, Schots A, Bakker J, Gommers F J, Jongsma M A, Bosch D, Steikema W J (1994) Coordinate expression of antibody subunit genes yields high levels of functional antibodies in roots of transgenic tobacco. Plant Mol. Biol. 26: 1701-1710.
73. Vaucheret H, Beclin C, Elmayan T, Feuerbach F, Godon C, Morel J B, Mourrain P, Palauqui J C, Vernhettes S (1998) Transgene-induced gene silencing in plants. Plant J 16: 651-659.
74. Verch T, Yusibov V, Koprowski H (1998) Expression and assembly of a full-length monoclonal antibody in plant using a plant virus vector. J Immunol. Meth. 220: 69-75.
75. Vierling E (1991) Annu. Rev. Plant Physiol. Plant Mol. Biol. 42: 579-620.
76. Walther-Larsen H., Brandt J., Collinge D. B., Thordal-Christensen H. (1993). A pathogen-induced gene of barley encodes a HSP90 homologue showing striking similarity to vertebrate forms resident in the endoplasmic reticulum. Plant Mol. Biol. 21:1097-1108.
77. Ye G N, Daniell H, Sanford J C (1990) Optimization of delivery of foreign DNA into higher plant chloroplasts. Plant Mol. Biol. 15:809-819.
78. Zeitlin L, Olmsted S S, Moench T R, Co M S, Martinell B J, Paradkar V M, Russell D V, Queen C, Cone R A, Whaley K J (1998) A humanized monoclonal antibody produced in plants for immunoprotection of the vagina against genital herpes. Nature Biotechnology. 16: 1361-1364.

We claim:

1. A vector useful for transformation of a target tobacco plastid, said vector comprising an expression cassette comprising as operably linked components, a promoter operative in the target plastid, a selectable marker sequence, at least one DNA sequence encoding an antibody, a transcription termination region functional in the target plastid, and on each of the 5′ and 3′ ends of said expression cassette DNA sequences which are homologous to a part of the target tobacco plastid genome.

2. The vector of claim 1, wherein said DNA sequence encodes a humanized antibody.

3. A method for producing a protein in a plastid, said method comprising:
introducing a plastid expression vector into a tobacco cell having a target plastid,
said plastid expression vector comprising as operably linked components
a DNA sequence containing at least one plastid replication origin functional in the target plastid,
a transcriptional initiation region functional in the target plastid,
at least one heterologous DNA sequence encoding an antibody, and
a transcriptional termination region functional in the target plasmid, whereby said heterologous DNA is introduced into the target plastid in the tobacco cell; and wherein said antibody is produced.

4. The method of claim 3, wherein said heterologous DNA sequence encodes a humanized antibody.

5. The method wherein said expression cassette comprises a heavy chain variable region fused to a constant region followed by a light chain variable region fused to a constant region.

6. A tobacco plastid comprising a DNA sequence encoding a polypeptide multimer encoding an immunologically active immunoglobulin molecule.

7. A tobacco plant cell comprising at least one plastid of claim 6.

8. A tobacco plant comprising at least one plastid of claim 6.

9. A tobacco plastid preparation comprising plastids of claim 6.

10. A transgenic tobacco plant that has been transformed by the vector of claim 1.

11. A progeny plant of the plant of claim 10 wherein said plant comprises said DNA sequence in a plastid genome of said progeny plant.

12. A seed produced by a plant of claim 8.

13. A composition comprising a polypeptide multimer and plant material, wherein said multimer comprises an immunologically active immunoglobulin prepared from plant plastids of claim 6.

14. The composition of claim 13 wherein said immunoglobulin molecule is non-glycosylated.

15. The composition of claim 13 wherein the DNA sequence encoding said immunoglobulin molecule comprises at least one sequence encoding a glycosylation signal sequence.

16. The composition of claim 13 wherein the polypeptide multimer further comprises a J chain.

17. The composition of claim 13 wherein the polypeptide multimer further comprises a secretory component.

18. The composition of claim 17 wherein the polypeptide multimer further comprises a J chain and secretory component.
